(19) United States
(12) Patent Application Publication
(10) Pub. No.: US 2007/0243215 A1
(43) Pub. Date: Oct. 18, 2007

(54) Adjuvant for Dna Vaccines

(76) Inventors: Richard L. Miller, Maplewood, MN (US); Mauro Provinciali, Ancona (IT); Arianna Smorlesi, Torrette (IT)

Correspondence Address: 3M INNOVATIVE PROPERTIES COMPANY, PO BOX 33427, ST. PAUL, MN, 55133-3427, US

(21) Appl. No.: 11/576,312

(22) PCT Filed: Oct. 7, 2005
(86) PCT No.: PCT/US05/36594

§ 371 Date: Mar. 29, 2007

Related U.S. Application Data
(60) Provisional application No. 60/617,014, filed on Oct. 8, 2004.
(60) Provisional application No. 60/688,540, filed on Jun. 8, 2005.

(51) Int. Cl. A61K 39/39; A61K 39/00; A61P 43/00

(52) U.S. Cl. 424/277.1; 424/278.1

(57) ABSTRACT

The present invention provides a DNA vaccine useful for treating breast cancer. Generally, the vaccine includes an expression vector that encodes a clinically relevant breast cancer-associated antigenic peptide and an IRM compound. The present invention also provides a DNA vaccine adjuvant that can increase the efficacy of a DNA vaccine. Generally, the adjuvant includes a TLR8-selective agonist.

### BACKGROUND

There has been a major effort in recent years, with significant success, to discover new drug compounds that act by stimulating certain key aspects of the immune system, as well as by suppressing certain other aspects (see, e.g., U.S. Pat. Nos. 6,039,969 and 6,200,592). These compounds, referred to herein as immune response modifiers (IRMs), appear to act through basic immune system mechanisms known as Toll-like receptors (TLRs) to induce selected cytokine biosynthesis. They may be useful for treating a wide variety of diseases and conditions. For example, certain IRMs may be useful for treating viral diseases (e.g., human papilloma virus, hepatitis, herpes), neoplasias (e.g., basal cell carcinoma, squamous cell carcinoma, actinic keratosis, melanoma), and T_{H}2-mediated diseases (e.g., asthma, allergic rhinitis, atopic dermatitis), and are also useful as vaccine adjuvants (U.S. Pat. No. 6,083,505 and U.S. Patent Publication No. US 2004/0076633).

Many of the IRM compounds are small organic molecule imidazoquinoline amine derivatives (see, e.g., U.S. Pat. No. 4,689,338), but a number of other compound classes are known as well (see, e.g., U.S. Pat. Nos. 5,446,153; 6,194,425; and 6,110,929; and International Publication Number WO 2005/079195) and more are still being discovered. Other IRMs have higher molecular weights, such as oligonucleotides, including CpGs (see, e.g., U.S. Pat. No. 6,194,388).

New and innovative treatment strategies, such as immunotherapies, are needed to improve outcomes in breast cancer, which too frequently recurs or progresses despite aggressive multimodality therapy. Cancer vaccines have the potential to treat existing cancer, prevent its recurrence, or both. In addition, breast cancer vaccines may be an ideal intervention for preventing ductal carcinoma in situ (DCIS), a very early form of breast cancer, from progressing to invasive cancer.

One treatment strategy involves the administration of a vaccine targeted against the HER-2/neu protein. This protein is found in abnormally high amounts on the cell surface of over 50% of DCIS tumors and 30% of invasive breast cancers. The HER-2/neu protein is found on the surface of the cells and receives signals that cause these cells to grow. When present at abnormally high levels, the HER-2/neu protein can cause a cell to respond too aggressively to growth signals, thus growing out of control and resulting in neoplastic transformation (i.e., tumor growth).

Trastuzumab (HERCEPTIN, Genentech, Inc.) is a monoclonal antibody directed against the HER-2/neu protein, and has been approved for the treatment of HER-2/neu-driven breast cancer. The monoclonal antibody is thought to bind to at least some of the HER-2/neu protein on the surface of tumor cells, thereby inhibiting the bound HER-2/neu from receiving growth signals. Also, when the antibody binds to the HER-2/neu protein, it may help the immune system identify the tumor cells as abnormal and, therefore, help target the tumor cells for destruction and/or elimination by cells of the immune system.

Genetic immunization against tumor antigens is another strategy for inducing an immune response able to oppose cancer progression. Genetic immunization involves vaccinating a subject with a DNA expression vector that encodes at least a portion of a tumor-specific antigen. Once vaccinated, cells in the subject's body can take up the expression vector and express genes encoded on the vector (e.g., tumor antigens). Expression of a tumor antigen off of the expression vector can prompt the subject's immune system to generate (a) antibodies against the tumor antigen and, therefore, tumor cells, and/or (b) antigen-specific cytotixic T lymphocytes (CTLs).

### SUMMARY

It has been found that certain IRM compounds can be useful as adjuvants for DNA vaccines.

Accordingly, the invention provides a DNA vaccine that includes an IRM compound and an expression vector that encodes a clinically relevant breast cancer-associated antigenic peptide. In some embodiments, the vaccine may be a single formulation, while in certain alternative embodiments, the expression vector and the IRM compound may be provided in separate formulations.

In another aspect, the invention provides a DNA vaccine adjuvant that includes a TLR8-selective agonist, and DNA vaccines that include a TLR8-selective agonist as an adjuvant.

In another aspect, the invention provides a method of treating breast cancer in a subject. Generally, the method includes administering to the subject an expression vector that encodes a clinically relevant breast cancer-associated antigenic peptide in an amount effective to generate an immune response against the clinically relevant breast cancer-associated antigenic peptide; and administering to the subject an IRM compound in an amount effective to potentiate the immune response to the clinically relevant breast cancer-associated antigenic peptide. In some embodiments, the breast cancer may include invasive breast cancer or ductal carcinoma in situ.

In yet another aspect, the invention provides the use of an IRM compound in the manufacture of a DNA vaccine for treating breast cancer in which the DNA vaccine includes an IRM compound and an expression vector that encodes a clinically relevant breast cancer-associated antigenic peptide.

In another aspect, the invention provides a method of treating cancer in a subject. Generally, the method includes administering to the subject an expression vector that encodes a clinically relevant cancer-associated antigenic peptide in an amount effective to generate an immune response against the clinically relevant cancer-associated antigenic peptide; and administering to the subject a TLR8-selective agonist in an amount effective to potentiate the immune response to the clinically relevant cancer-associated antigenic peptide. In some embodiments, the cancer may include breast cancer, hepatocellular cancer, cervical cancer, colon cancer, melanoma, or lung cancer.

In yet another aspect, the invention provides the use of an IRM compound in the manufacture of a DNA vaccine for treating cancer in which the DNA vaccine includes a TLR8-selective agonist and an expression vector that encodes a clinically relevant cancer-associated antigenic peptide.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 shows that an IRM, as an adjuvant with a HER-2/neu-based breast cancer DNA vaccine, increases the vaccine's efficacy as measured by preventing tumors (FIG. 1a) and reducing the number of tumors (FIG. 1b).

FIG. 2 shows that another IRM, as an adjuvant with a HER-2/neu-based breast cancer DNA vaccine, increases the vaccine's efficacy as measured by preventing tumors (FIG. 2a) and reducing the number of tumors (FIG. 2b).

FIG. 3 shows that IRM compounds, as adjuvants with a HER-2/neu-based breast cancer DNA vaccine, increase antigen-specific humoral immunity induced by the vaccine.

FIG. 4 shows that IRM compounds, as adjuvants with a HER-2/neu-based breast cancer DNA vaccine, increase cytotoxicity induced by the vaccine.

FIG. 5 shows that IRM compounds, as adjuvants with a HER-2/neu-based breast cancer DNA vaccine, increase the percentage of cells that are induced by the vaccine to produce anti-tumor cytokines IFN-γ (FIG. 5a), IL-2 (FIG. 5b), and IL-10 (FIG. 5c).

FIG. 6 shows that serum from mice treated with an IRM and a HER-2/neu-based breast cancer DNA vaccine can provide protection against tumor development in recipient mice.

### DETAILED DESCRIPTION OF ILLUSTRATIVE EMBODIMENTS OF THE INVENTION

Certain IRM compounds have been identified as being useful as adjuvants for DNA vaccines that target a clinically relevant cancer-associated antigenic peptide. Moreover, while certain IRM compounds have been suggested as possible DNA vaccine adjuvants, this is the first demonstration that an IRM compound can be effective as an adjuvant for a DNA vaccine that targets a spontaneously arising (i.e., non-transfected) tumor-specific antigen.

For the purposes of the present invention, the following terms shall have the following meanings:

“Agonist” refers to a compound that can combine with a receptor (e.g., a TLR) to induce a cellular activity. An agonist may be a ligand that directly binds to the receptor. Alternatively, an agonist may combine with a receptor indirectly by, for example, (a) forming a complex with another molecule that directly binds to the receptor, or (b) otherwise results in the modification of another compound so that the other compound directly binds to the receptor. An agonist may be referred to as an agonist of a particular TLR (e.g., a TLR8 agonist) or a particular combination of TLRs (e.g., a TLR 7/8 agonist—an agonist of both TLR7 and TLR8).

“Antigen” refers to any substance that is capable of being the target of an immune response. An antigen may be the target of, for example, a cell-mediated and/or humoral immune response raised by a subject organism. Alternatively, an antigen may be the target of a cellular immune response (e.g., immune cell maturation, production of cytokines, production of antibodies, etc.) when contacted with an immune cell.

“Antigenic peptide” refers to a peptide of any length, derived from the indicated protein, that is capable of being the target of a cell-mediated and/or humoral immune response. For example, “antigenic HER-2/neu peptide” refers to a peptide derived from human, rat, or mouse HER-2/neu protein, that is capable of being the target of a cell-mediated and/or humoral immune response. As another example, “antigenic mammaglobulin-A” peptide refers to a peptide derived from mammaglobulin-A that is capable of being the target of a cell-mediated and/or humoral immune response.

“DNA vaccine” and variations thereof refer to a nucleotide sequence that encodes an antigenic peptide and may be directly introduced into a subject to induce an immune response in the subject against the antigenic peptide.

“HER-2“, “neu”, and “HER-2/neu” refer, interchangeably, to a 185 kD protein encoded by the rat neu proto-oncogene and its human homolog, HER-2, or its murine homolog, neu.

“Peptide” refers to a sequence of amino acid residues without regard to the length of the sequence. Therefore, the term “peptide” refers to any amino acid sequence having at least two amino acids and includes full-length proteins and, as the case may be, polyproteins.

In one aspect, the invention provides a DNA vaccine for treating breast cancer. Generally, the vaccine includes an expression vector that encodes a clinically relevant breast cancer-associated antigenic peptide and an IRM compound.

As used herein, “treating,” or “to treat” a condition refers to reducing, limiting progression, ameliorating, or resolving, to any extent, a symptom or clinical sign related to a condition. A “treatment” refers to any substance, composition, regimen, etc. that is capable of treating a condition, and may be described as therapeutic, prophylactic, or both. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition.

As used herein, a “clinically relevant breast cancer-associated antigenic peptide” refers to a cell marker, typically a peptide or full-length protein, that is both (a) differentially expressed between normal cells and tumor cells, and (b) the differential expression can be exploited to treat or prevent occurrence of breast cancer.

Differential expression between normal cells and tumor cells generally means that tumor cells express the marker to a greater extent than normal cells do. For example, some clinically relevant breast cancer-associated antigenic peptides may be expressed by tumor cells but not expressed in normal cells. Such antigenic peptides may be considered tumor-specific antigenic peptides because they are expressed only—i.e., specifically—by tumor cells. In other cases, however, a clinically relevant breast cancer-associated antigenic peptide may be naturally expressed by normal cells, but overexpressed—i.e., expressed at a greater than normal level—by tumor cells.

An expression vector may be of any suitable form including, but not limited to, naked DNA. Alternatively, the expression vector may be packaged such as, for example, in, or as part of, an attenuated bacterium or virus-derived vector such as, for example, an alphavirus vector such as those based upon Sindbid virus, Semliki Forest virus (SFV), and Venezuelan equine encephalitis virus (VEE). Suitable alphavirus vectors include, for example, double promoter vectors and replicon vectors such as those described, for example, in Leitner et al., Nature Medicine (2003), vol. 9, pp. 33-39; Dubensky et al., J. Virol. (1996), vol. 70, pp. 508-519; and Pushko et al., Virol. (1997), vol. 239, pp. 389-401.

Expression vectors that encode a clinically relevant breast cancer-associated antigenic peptides are known. For example, pCMVneuNT encodes full-length rat neu protein. Certain evidence suggests, however, that expression vectors that encode truncated forms of HER-2/neu may be more effective at inducing protective antitumor immunity than vectors that encode full-length neu protein. Expression vectors that encode truncated forms of HER-2/neu include, for example, pCMV-ECD (encoding the neu extracellular domain), and pCMV-ECD-TM (encoding the neu extracellular and transmembrane domains). Expression vectors that encode at least a portion of HER-2/neu are described, for example, in Chen, Y. et al., Cancer Research (1998), vol. 58, pp. 1965-1971.

Mammaglobulin-A is another clinically relevant breast cancer-associated antigenic peptide, expressed in 80% of breast tumors. Mice vaccinated with mammaglobulin-A cDNA can generate a CD8⁺ cytotoxic T lymphocyte (CTL) response against mammaglobulin-A⁺ tumors. Moreover, transfer of CD8⁺ CTLs from vaccinated mice to animals with actively growing mammaglobulin-A⁺ tumors caused significant tumor regression. Certain mammaglobulin-A epitopes have been recognized by CD8⁺ CTLs from both immunized mice and breast cancer patients. Expression vectors that encode at least a portion of mammaglobulin-A are described, for example, in Narayanan, K. et al., J. Natl. Cancer Inst. (2004), vol. 96, pp. 1388-1396).

MUC1 (polymorphic epithelial mucin, or PEM) is another clinically relevant breast cancer-associated antigenic peptide. MUC1 is expressed by tumor cells of many cancers such as, for example, most epithelial cancers. The MUC1 mucin is a high-molecular-weight (>400 kD) transmembrane glycoprotein that is expressed at the apical cell surface of normal glandular epithelia and overexpressed in certain cancers such as, for example, breast cancer. Cytotoxic T lymphocytes (CTLs) that recognize MUC1 core peptides and mediate lysis of tumor targets in vitro have been obtained from patients with breast, pancreatic, and ovarian carcinomas. Circulating MUC1 immunoglobulin M (IgM) antibodies have been found in patients with breast, colon, and pancreatic cancer. Circulating MUC1 immunoglobulin G (IgG) antibodies have been detected in patients with colorectal cancer. Mice vaccinated with an expression vector encoding at least a portion of MUC1 are protected against tumor development after subsequent challenge with MUC1-expressing syngenic tumor cells. Certain expression vectors encoding at least a portion of MUC1 can generate specific CD4⁺ and CD8⁺ T cell response in vivo after challenge with MUC1-expressing tumor cells. Expression vectors that encode at least a portion of MUC1 are described, for example, in Plunkett, T. et al., Int. J. Cancer (2004), vol. 109, pp. 691-697.

Other expression vectors that encode a clinically relevant breast cancer-associated antigenic peptide include, for example, SINCP-βgal (Chiron Corp., Emeryville, Calif.) and certain VEEreplicon particles (VRP, AlphaVax, Inc., Research Triangle park, N.C.).

Accordingly, in one embodiment, the vaccine includes (a) an expression vector that encodes an antigenic HER-2/neu peptide, and (b) an IRM compound. In other embodiments, the vaccine includes (a) an expression vector that encodes an antigenic mammaglobulin-A peptide, and (b) an IRM compound. In another embodiment, the vaccine includes (a) an expression vector that encodes an antigenic MUC1 peptide, and (b) an IRM compound. In another embodiment, the vaccine includes SINCP-βgal and an IRM compound. In yet another embodiment, the vaccine includes (a) a VEE replicon that encodes a breast cancer-associated antigenic peptide, and (b) an IRM compound.

In another aspect, the invention provides an adjuvant for use in a DNA vaccine, and the resulting DNA vaccines that include such an adjuvant. Generally, the adjuvant includes an IRM compound that is a TLR8-selective agonist. Thus, a DNA vaccine generally includes an expression vector that encodes a clinically relevant cancer-associated antigenic peptide, and an IRM compound that is a TLR8-selective agonist.

The adjuvant effect provided by the TLR8-selective agonist may not be vaccine-dependent. That is, a TLR8-selective agonist may be an effective adjuvant for any DNA vaccine that includes an expression vector that encodes any clinically relevant cancer-associated antigenic peptide. Thus, the description of certain clinically relevant cancer-associated antigenic peptides and expression vectors that encode such peptides is merely exemplary and not intended to be an exhaustive description of all suitable clinically relevant cancer-associated antigenic peptides and expression vectors that encode such peptides.

Clinically relevant cancer-associated antigenic peptides include those described above that are breast cancer-associated antigenic peptides, although some, such as, for example, MUC1, may be further associated with cancers other than breast cancer.

Alphafetoprotein (AFP) is a clinically relevant antigenic peptide associated with hepatocellular cancer (HCC). HCC, a primary liver cancer, is a major cause of cancer death. Endemic to Asia, the disease is prominent in individuals suffering from liver cirrhosis as a result of Hepatitis B infection. Approximately 1.2 million new cases arise annually and almost all those afflicted die within six months of diagnosis. Mice immunized with an expression vector encoding an antigenic portion of AFP experienced a delay in tumor growth. Such expression vectors are described, for example, in U.S. Patent Publication No. 2003/0143237.

Human papillomavirus (HPV) oncoproteins E6 and E7 are clinically relevant antigenic peptides associated with cervical cancer. HPV is present in most cervical cancers and the HPV oncoproteins E6 and E7 are consistently expressed in HPV-associated cancer cells and are responsible for their malignant transformation. Mice immunized with an expression vector that encodes an antigenic E7 peptide can generate an E7-specific CD8⁺ T lymphocyte immune response. Mice immunized with an expression vector that encodes an antigenic E6 peptide (a) can generate an E6-specific CD8⁺ T lymphocyte immune response, and (b) can be protected from tumor development after challenge with an E6-expressing tumor cell line. Expression vectors that encode at least an antigenic portion of E7 are described, for example, in Cheng, W. F., et al., J. Clin. Investig. (2001), vol. 108, pp. 669-678. Expression vectors that encode at least an antigenic portion of E6 are described, for example, in Peng et al. (2004) J.Virol. 78.16:8468-8476.

Tyrosinase-related protein-1 (TRP-1) is a clinically relevant antigenic peptide associated with melanoma. TRP-1 is a tumor rejection antigen expressed in high levels in melanoma cells. Mice immunized with expression vectors that encode at least a portion of TRP-1 were protected from the development of tumors after challenge with melanoma cells. Expression vectors encoding at least an antigenic portion of TRP-1 are described, for example, in Leitner et al. (2003), Nature Medicine, vol. 9, no. 1, pp. 33-39.

Vascular endothelial growth factor receptor 2 (VEGF2) is a clinically relevant antigenic peptide associated with many types of tumors. VEGF2 expression is upregulated during angiogenesis of tumor vasculature. Angiogenesis has a central role in the invasion, growth, and metastasis of solid tumors. Thus, an immune response against proliferating endothelial cells—those that overexpress VEGF2—in the tumor vasculature can cause the collapse of tumor vessels, thereby essentially starving the cancer before it can fully develop. Mice vaccinated with an expression vector encoding VEGF2 experienced inhibited tumor growth when challenged with melanoma or non-small cell lung carcinoma cells; were protected against spontaneous pulmonary metastases (e.g., non-small cell lung carcinoma); had prolonged lifespans after challenge with colon carcinoma cells; and, in a therapeutic model, experienced reduced growth of established metastases arising from colon carcinoma cells. Expression vectors encoding at least an antigenic portion of VEGF2 are described, for example, in Niethammer et al. (2002), Nature Medicine, vol. 8, no. 12, pp. 1369-1375.

Accordingly, in some embodiments the vaccine can include an expression vector that encodes a clinically relevant breast cancer-associated antigenic peptide—i.e., a HER-2/neu peptide, mammaglobulin-A peptide, or MUC1, and a TLR8-selective agonist. In other embodiments, the vaccine can include an expression vector that encodes a clinically relevant cancer-associated antigenic peptide such as, for example, an antigenic alphafetoprotein peptide (HCC-associated), an antigenic TRP-1 peptide (melanoma-associated), an antigenic VEGF2 peptide (multi-tumor-associated), or an antigenic E6 or E7 peptide (cervical cancer-associated), and a TLR8-selective agonist.

As described above, administering to a subject a DNA vaccine according to the invention can provide the subject with prophylactic and/or therapeutic cancer treatment. In another aspect, however, the invention provides a method of preparing a cancer treatment composition that can provide prophylactic and/or therapeutic cancer treatment to another. Generally, the method includes administering to a subject an IRM compound and an expression vector that encodes a clinically relevant cancer-associated antigenic peptide, permitting the subject to generate a serum immune response to the clinically relevant cancer-associated antigenic peptide, and, finally, collecting at least a portion of the subject's serum. The material collected from the subject may be further processed to enrich the collected material for certain substances (e.g., antibodies directed against the clinically relevant cancer-associated antigenic peptide) or deplete the collected material of certain substances (e.g., cells, ABO blood type antibodies, Rh factor).

At least a portion of the material collected from the subject (whether further processed or not) may be administered to a second subject in need of treatment of cancer associated with the clinically relevant cancer-associated antigenic peptide—e.g., one who is at risk of developing or has been diagnosed as having cancer associated with the clinically relevant cancer-associated antigenic peptide. Thus administering a DNA vaccine of the invention may provide either primary treatment (i.e., to a subject to whom the DNA vaccine is administered), or secondary treatment (e.g., to a subject who receives serum collected from one to whom the DNA vaccine is administered).

IRM compounds include compounds that possess potent immunomodulating activity including but not limited to antiviral and antitumor activity. Certain IRMs modulate the production and secretion of cytokines. For example, certain IRM compounds induce the production and secretion of cytokines such as, e.g., Type I interferons, TNF-α, IL-1, IL-6, IL-8, IL-10, IL-12, MIP-1, and/or MCP-1. Additionally, some IRM compounds are said to suppress IL-1 and TNF (U.S. Pat. No. 6,518,265).

Certain IRMs are small organic molecules (e.g., molecular weight under about 1000 Daltons, preferably under about 500 Daltons, as opposed to large biological molecules such as proteins, peptides, and the like) such as those disclosed in, for example, U.S. Pat. Nos. 4,689,338; 4,929,624; 5,266,575; 5,268,376; 5,346,905; 5,352,784; 5,389,640; 5,446,153; 5,482,936; 5,756,747; 6,110,929; 6,194,425; 6,331,539; 6,376,669; 6,451,810; 6,525,064; 6,541,485; 6,545,016; 6,545,017; 6,573,273; 6,656,938; 6,660,735; 6,660,747; 6,664,260; 6,664,264; 6,664,265; 6,667,312; 6,670,372; 6,677,347; 6,677,348; 6,677,349; 6,683,088; 6,756,382; 6,797,718; and 6,818,650; U.S. Patent Publication Nos. 2004/0691491; 2004/0147543; and 2004/0176367; and International Publication Nos. WO 2005/18551, WO 2005/18556, WO 2005/20999, WO 2005/032484, WO 2005/048933, WO 2005/048945, WO 2005/051317, WO 2005/051324, WO 2005/066169, WO 2005/066170, WO 2005/066172, WO 2005/076783, and WO 2005/079195.

Additional examples of small molecule IRMs include certain purine derivatives (such as those described in U.S. Pat. Nos. 6,376,501, and 6,028,076), certain imidazoquinoline amide derivatives (such as those described in U.S. Pat. No. 6,069,149), certain imidazopyridine derivatives (such as those described in U.S. Pat. No. 6,518,265), certain benzimidazole derivatives (such as those described in U.S. Pat. No. 6,387,938), certain derivatives of a 4-aminopyrimidine fused to a five membered nitrogen containing heterocyclic ring (such as adenine derivatives described in U.S. Pat. Nos. 6,376,501; 6,028,076 and 6,329,381; and in WO 02/08905), and certain 3-β-D-ribofuranosylthiazolo[4,5-d]pyrimidine derivatives (such as those described in U.S. Publication No. 2003/0199461).

Other IRMs include large biological molecules such as oligonucleotide sequences. Some IRM oligonucleotide sequences contain cytosine-guanine dinucleotides (CpG) and are described, for example, in U.S. Pat. Nos. 6,194,388; 6,207,646; 6,239,116; 6,339,068; and 6,406,705. Some CpG-containing oligonucleotides can include synthetic immunomodulatory structural motifs such as those described, for example, in U.S. Pat. Nos. 6,426,334 and 6,476,000. Other IRM nucleotide sequences lack CpG sequences and are described, for example, in International Patent Publication No. WO 00/75304.

Other IRMs include biological molecules such as aminoalkyl glucosaminide phosphates (AGPs) and are described, for example, in U.S. Pat. Nos. 6,113,918; 6,303,347; 6,525,028; and 6,649,172.

Unless otherwise indicated, reference to a compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers.

In some embodiments of the present invention, the IRM compound may be an agonist of at least one TLR, preferably an agonist of TLR6, TLR7, or TLR8. The IRM may also in some cases be an agonist of TLR4 or TLR9. In some embodiments of the present invention, the IRM compound may be a small molecule immune response modifier (e.g., molecular weight of less than about 1000 Daltons).

In some embodiments of the present invention, the IRM compound may include a 2-aminopyridine fused to a five membered nitrogen-containing heterocyclic ring, or a 4-aminopyrimidine fused to a five membered nitrogen-containing heterocyclic ring.

IRM compounds suitable for use in the invention include compounds having a 2-aminopyridine fused to a five membered nitrogen-containing heterocyclic ring. Such compounds include, for example, imidazoquinoline amines including but not limited to substituted imidazoquinoline amines such as, for example, amide substituted imidazoquinoline amines, sulfonamide substituted imidazoquinoline amines, urea substituted imidazoquinoline amines, aryl ether substituted imidazoquinoline amines, heterocyclic ether substituted imidazoquinoline amines, amido ether substituted imidazoquinoline amines, sulfonamido ether substituted imidazoquinoline amines, urea substituted imidazoquinoline ethers, thioether substituted imidazoquinoline amines, hydroxylamine substituted imidazoquinoline amines, oxime substituted imidazoquinoline amines, 6-, 7-, 8-, or 9-aryl, heteroaryl, aryloxy or arylalkyleneoxy substituted imidazoquinoline amines, and imidazoquinoline diamines; tetrahydroimidazoquinoline amines including but not limited to amide substituted tetrahydroimidazoquinoline amines, sulfonamide substituted tetrahydroimidazoquinoline amines, urea substituted tetrahydroimidazoquinoline amines, aryl ether substituted tetrahydroimidazoquinoline amines, heterocyclic ether substituted tetrahydroimidazoquinoline amines, amido ether substituted tetrahydroimidazoquinoline amines, sulfonamido ether substituted tetrahydroimidazoquinoline amines, urea substituted tetrahydroimidazoquinoline ethers, thioether substituted tetrahydroimidazoquinoline amines, hydroxylamine substituted tetrahydroimidazoquinoline amines, oxime substituted tetrahydroimidazoquinoline amines, and tetrahydroimidazoquinoline diamines; imidazopyridine amines including but not limited to amide substituted imidazopyridine amines, sulfonamide substituted imidazopyridine amines, urea substituted imidazopyridine amines, aryl ether substituted imidazopyridine amines, heterocyclic ether substituted imidazopyridine amines, amido ether substituted imidazopyridine amines, sulfonamido ether substituted imidazopyridine amines, urea substituted imidazopyridine ethers, and thioether substituted imidazopyridine amines; 1,2-bridged imidazoquinoline amines; 6,7-fused cycloalkylimidazopyridine amines; imidazonaphthyridine amines; tetrahydroimidazonaphthyridine amines; oxazoloquinoline amines; thiazoloquinoline amines; oxazolopyridine amines; thiazolopyridine amines; oxazolonaphthyridine amines; thiazolonaphthyridine amines; pyrazolopyridine amines; pyrazoloquinoline amines; tetrahydropyrazoloquinoline amines; pyrazolonaphthyridine amines; tetrahydropyrazolonaphthyridine amines; and 1H-imidazo dimers fused to pyridine amines, quinoline amines, tetrahydroquinoline amines, naphthyridine amines, or tetrahydronaphthyridine amines

In one embodiment, the IRM compound may be an imidazoquinoline amine such as, for example, 1-(2-methylpropyl)-1H-imidazo[4,5-c]quinolin-4-amine or 4-amino-α,α,2-trimethyl-1H-imidazo[4,5-c]quinoline-1-ethanol.

In alternative embodiments, the IRM compound may be a thiazoloquinoline amine, a thiazolopyridine amine, or a thiazolonaphthyridine amine. In one particular embodiment, the IRM compound may be, for example, 2-propylthiazolo[4,5-c]quinolin-4-amine. In another embodiment, the IRM compound may be, for example, 2-propyl-7-(pyridin-3-yl)-thiazolo[4,5-c]quinolin-4-amine. In another embodiment, the IRM compound may be, for example, [3-(4-amino-2-propylthiazolo[4,5-c]quinolin-7-yl)phenyl]methanol. In yet another embodiment, the IRM compound may be, for example, N-[3-(4-amino-2-propylthiazolo[4,5-c]quinolin-7-yl)phenyl]methanesulfonamide.

In certain embodiments, the IRM compound may be an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, or a thiazolonaphthyridine amine.

In certain embodiments, the IRM compound may be a a substituted imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

As used herein, a substituted imidazoquinoline amine refers to an amide substituted imidazoquinoline amine, a sulfonamide substituted imidazoquinoline amine, a urea substituted imidazoquinoline amine, an aryl ether substituted imidazoquinoline amine, a heterocyclic ether substituted imidazoquinoline amine, an amido ether substituted imidazoquinoline amine, a sulfonamido ether substituted imidazoquinoline amine, a urea substituted imidazoquinoline ether, a thioether substituted imidazoquinoline amine, a hydroxylamine substituted imidazoquinoline amine, an oxime substituted imidazoquinoline amine, a 6-, 7-, 8-, or 9-aryl, heteroaryl, aryloxy or arylalkyleneoxy substituted imidazoquinoline amine, or an imidazoquinoline diamine. As used herein, substituted imidazoquinoline amines specifically and expressly exclude 1-(2-methylpropyl)-1H-imidazo[4,5-c]quinolin-4-amine and 4-amino-α,α-dimethyl-2-ethoxymethyl-1H-imidazo[4,5-c]quinolin-1-ethanol.

Suitable IRM compounds also may include the purine derivatives, imidazoquinoline amide derivatives, benzimidazole derivatives, adenine derivatives, aminoalkyl glucosaminide phosphates, and oligonucleotide sequences described above.

In some embodiments, the IRM compound may be a compound identified as an agonist of one or more TLRs. For example, the IRM compound may be an agonist of TLR8. In certain embodiments, the IRM compound may be a TLR8-selective agonist. As used herein, the term “TLR8-selective agonist” refers to any compound that acts as an agonist of TLR8, but does not act as an agonist of TLR7. A “TLR7/8 agonist” refers to a compound that acts as an agonist of both TLR7 and TLR8.

A TLR8-selective agonist may act as an agonist of TLR8 and one or more of TLR1, TLR2, TLR3, TLR4, TLR5, TLR6, TLR9, or TLR10, but not TLR7. Accordingly, while “TLR8-selective agonist” may refer to a compound that acts as an agonist for TLR8 and for no other TLR, it may alternatively refer to a compound that acts as an agonist of TLR8 and, for example, TLR4.

The TLR agonism for a particular compound may be assessed in any suitable manner. For example, assays and recombinant cell lines suitable for detecting TLR agonism of test compounds are described, for example, in U.S. Patent Publication Nos. US2004/0014779, US2004/0132079, US2004/0162309, US2004/0171086, US2004/0191833, and US2004/0197865.

Regardless of the particular assay employed, a compound can be identified as an agonist of a particular TLR (e.g., TLR8) if performing the assay with a compound results in at least a threshold increase of some biological activity mediated by the particular TLR. Conversely, a compound may be identified as not acting as an agonist of a specified TLR (e.g., TLR7) if, when used to perform an assay designed to detect biological activity mediated by the specified TLR, the compound fails to elicit a threshold increase in the biological activity. Unless otherwise indicated, an increase in biological activity refers to an increase in the same biological activity over that observed in an appropriate control. An assay may or may not be performed in conjunction with the appropriate control. With experience, one skilled in the art may develop sufficient familiarity with a particular assay (e.g., the range of values observed in an appropriate control under specific assay conditions) that performing a control may not always be necessary to determine the TLR agonism of a compound in a particular assay.

The precise threshold increase of TLR-mediated biological activity for determining whether a particular compound is or is not an agonist of a particular TLR in a given assay may vary according to factors known in the art including but not limited to the biological activity observed as the endpoint of the assay, the method used to measure or detect the endpoint of the assay, the signal-to-noise ratio of the assay, the precision of the assay, and whether the same assay is being used to determine the agonism of a compound for both TLRs. Accordingly it is not practical to set forth generally the threshold increase of TLR-mediated biological activity required to identify a compound as being an agonist or a non-agonist of a particular TLR for all possible assays. Those of ordinary skill in the art, however, can readily determine the appropriate threshold with due consideration of such factors.

Assays employing HEK293 cells transfected with an expressible TLR structural gene may use a threshold of, for example, at least a three-fold increase in a TLR-mediated biological activity (e.g., NFκB activation) when the compound is provided at a concentration of, for example, from about 1 μM to about 10 μM for identifying a compound as an agonist of the TLR transfected into the cell. However, different thresholds and/or different concentration ranges may be suitable in certain circumstances. Also, different thresholds may be appropriate for different assays.

Each of the IRM compound and expression vector may be provided in any formulation suitable for administration to a subject. Suitable types of formulations are described, for example, in U.S. Pat. No. 5,238,944; U.S. Pat. No. 5,939,090; U.S. Pat. No. 6,245,776; European Patent No. EP 0 394 026; and U.S. Patent Publication Nos. 2003/0199538 and 2004/0076633. Suitable formulations may include, but are not limited to, a solution, a suspension, an emulsion, or any form of mixture. A suitable formulation may include any pharmaceutically acceptable excipient, carrier, or vehicle. A suitable formulation for delivering the expression vector may include the expression vector as naked DNA. Alternatively, the expression vector may be packaged such as, for example, in, or as part of, a virus-derived replicon or attenuated bacterium.

A formulation containing the DNA vaccine and/or adjuvant IRM compound may be administered in any suitable manner such as, for example, non-parenterally or parenterally. As used herein, non-parenterally refers to administration through the digestive tract, including by oral ingestion. Parenterally refers to administration other than through the digestive tract such as, for example, intravenously, intramuscularly, transdermally, subcutaneously, transmucosally (e.g., by inhalation), or topically.

The expression vector and the IRM compound may be provided together in a single formulation. Alternatively, the expression vector and the IRM compound may be provided separately in different formulations. When provided in separate formulations, the expression vector and the IRM compound may be administered at a single site or at different sites, by the same or different routes, and at the same or at different times.

The composition of a formulation that includes the IRM compound may vary according to factors known in the art including but not limited to the physical and chemical nature of the IRM compound, the nature of the carrier, the intended dosing regimen, the state of the subject's immune system (e.g., suppressed, compromised, stimulated), the method of administering the IRM compound, and the potency of the DNA vaccine. Accordingly, it is not practical to set forth generally the composition of a formulation effective for use as a DNA vaccine adjuvant for all possible applications. Those of ordinary skill in the art, however, can readily determine an appropriate formulation with due consideration of such factors.

In some embodiments, the formulation can include, for example, from about 0.0001% to about 10% (unless otherwise indicated, all percentages provided herein are weight/weight with respect to the total formulation) IRM compound, although in some embodiments the formulation may include IRM compound in a concentration outside of this range. In certain embodiments, the formulation includes from about 0.01% to about 5% IRM compound, for example, a formulation that includes from about 0.1 % to about 1.0% IRM compound.

An amount of an IRM compound effective for use as a DNA vaccine adjuvant is an amount sufficient to increase the efficacy of the DNA vaccine. Efficacy of a DNA vaccine may be indicated by, for example, one or more of the following: induction of certain cytokines (e.g., TNF-α, IL-12, IFN-γ, IFN-α, MCP-1, IP-10), increasing humoral titers of antibodies directed against an antigen encoded by the DNA vaccine, reducing the number or size of tumors, delaying the incidence of tumors, prolonging the expected lifespan of the subject, generating antigen-specific CTLs, and/or upregulating co-stimulatory marker expression on antigen presenting cells (APCs), especially, for example, DC-1 cells.

The precise amount of IRM compound effective for use as a DNA vaccine adjuvant may vary according to factors known in the art including but not limited to the physical and chemical nature of the IRM compound, the nature of the carrier, the intended dosing regimen, the state of the subject's immune system (e.g., suppressed, compromised, stimulated), the method of administering the IRM compound, and the potency of the DNA vaccine. Accordingly, it is not practical to set forth generally the amount that constitutes an amount of IRM compound effective for use as a DNA vaccine adjuvant for all possible applications. Those of ordinary skill in the art, however, can readily determine the appropriate amount with due consideration of such factors.

In some embodiments, the IRM compound may be provided in a dose of, for example, from about 100 ng/kg to about 50 mg/kg, although in some embodiments the IRM compound may be provided in a dose outside this range. In some of these embodiments, the IRM compound may be provided in a dose of from about 10 μg/kg to about 5 mg/kg, for example, a dose of about 0.6 mg/kg.

The dosing regimen may depend at least in part on many factors known in the art including but not limited to the physical and chemical nature of the IRM compound, the nature of the carrier, the amount of IRM being administered, the state of the subject's immune system (e.g., suppressed, compromised, stimulated), the method of administering the IRM compound, and the potency and method of delivery of the DNA vaccine. Accordingly it is not practical to set forth generally the dosing regimen effective for increasing the efficacy of a DNA vaccine for all possible applications. Those of ordinary skill in the art, however, can readily determine an appropriate dosing regimen with due consideration of such factors.

In some embodiments of the invention, the IRM compound may be administered, for example, once to about once daily, although in some embodiments the IRM compound may be administered at a frequency outside this range. In certain embodiments, the IRM compound may be administered from about once per week to about once per day. In one particular embodiment, the IRM compound is administered once every three days.

The methods of the present invention may be performed on any suitable subject. Suitable subjects include but are not limited to animals such as but not limited to humans, non-human primates, rodents, dogs, cats, horses, pigs, sheep, goats, or cows.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

IRM Compounds

The IRM compounds used in the examples are shown in Table 1.

TABLE 1

| Compound | Chemical Name | Reference |
|---|---|---|
| IRM1 | 4-amino-α,α,2-trimethyl-1H-imidazo[4,5- | U.S. Pat. No. 5,266,575 |
| | c]quinoline-1-ethanol | Example C1 |
| IRM2 | 1-(2-methylpropyl)-1H-imidazo[4,5-c]quinolin-4- | U.S. Pat. No. 4,689,338 |
| | amine | Example 99 |
| IRM3 | 2-propylthiazolo[4,5-c]quinolin-4-amine | U.S. Pat. No. 6,110,929 |
| | | Example 12 |
| IRM4 | 2-propyl-7-(pyridin-3-yl)-thiazolo[4,5-c]quinolin- | U.S. Ser. No. |
| | 4-amine | 60/581205^{#} |
| IRM5 | N-[3-(4-amino-2-propylthiazolo[4,5-c]quinolin-7- | U.S. Ser. No. |
| | yl)phenyl]methanesulfonamide | 60/581205 |
| | | Example 2 |
| IRM6 | [3-(4-amino-2-propylthiazolo[4,5-c]quinolin-7- | U.S. Ser. No. |
| | yl)phenyl]methanol | 60/581205 |
| | | Example 1 |

^{#}This compound is not specifically exemplified but can be readily prepared using the synthetic methods disclosed in the cited reference.

### Example 1

### In vivo Tumor Growth

Female FVB/N mice, containing the activated rat neu gene (Charles River Laboratories, Hollister, Calif.) were maintained under specific-pathogen-free conditions and under standard light/dark regimen (12 hours light: 12 hours dark). Mice were housed in plastic non-galvanized cages (4-6 mice per cage) and fed with standard pellet food and tap water ad libitum.

IRM solutions were prepared by dissolving an IRM compound in 0.2% DMSO and water until the indicated final concentration was obtained.

The plasmid pCMV-ECD-TM, which encodes extracellular and transmembrane HER-2/neu regions under the control of the CMV eukaryotic promoter, has been described (Chen, Y. et al., Cancer Research (1998), vol. 58, pp. 1965-1971). Large scale preparation of plasmid DNA was performed using a Plasma Giga kit (Qiagen, Inc., Valencia, Calif.) according to the manufacturer's instructions.

Animals were divided into treatment groups (n=15) as follows: HER-2/neu +IRM1 (immunized with pCMV-ECD-TM, treated with IRM1), HER-2/neu +IRM2 (immunized 20 with pCMV-ECD-TM, treated with IRM2), Control (not immunized, not treated with IRM), HER-2/neu (immunized with pCMV-ECD-TM, not treated with IRM), IRM1 (not immunized, treated with IRM1), and IRM2 (not immunized, treated with IRM2).

Animals immunized with pCMV-ECD-TM DNA were immunized by particle-mediated immunotherapeutic delivery using a HELIOS gene gun system (Bio-Rad Laboratories, Inc., Hercules, Calif.) at eight, ten, and twelve weeks of age. Each vaccination included 2 μg plasmid DNA (two gene gun shots), administered according to manufacturer's instructions.

Animals treated with an IRM compound received 0.6 mg/kg of compound in 200 μL of water intraperitoneally. Those receiving IRM compound were treated every three days during the period of immunization (8-12 weeks of age), starting two days before the first DNA injection.

Incidence and growth of tumors were evaluated twice weekly by measuring neoplastic masses with calipers in two perpendicular diameters. Mice were classified as tumor bearers if they developed a tumor having a mean diameter of at least 3 mm. Mice with no evidence of tumors at the end of the evaluation period were classified as tumor-free. The mean number of palpable mammary carcinomas per mouse was calculated as (cumulative number of incident tumors)/(total number of mice).

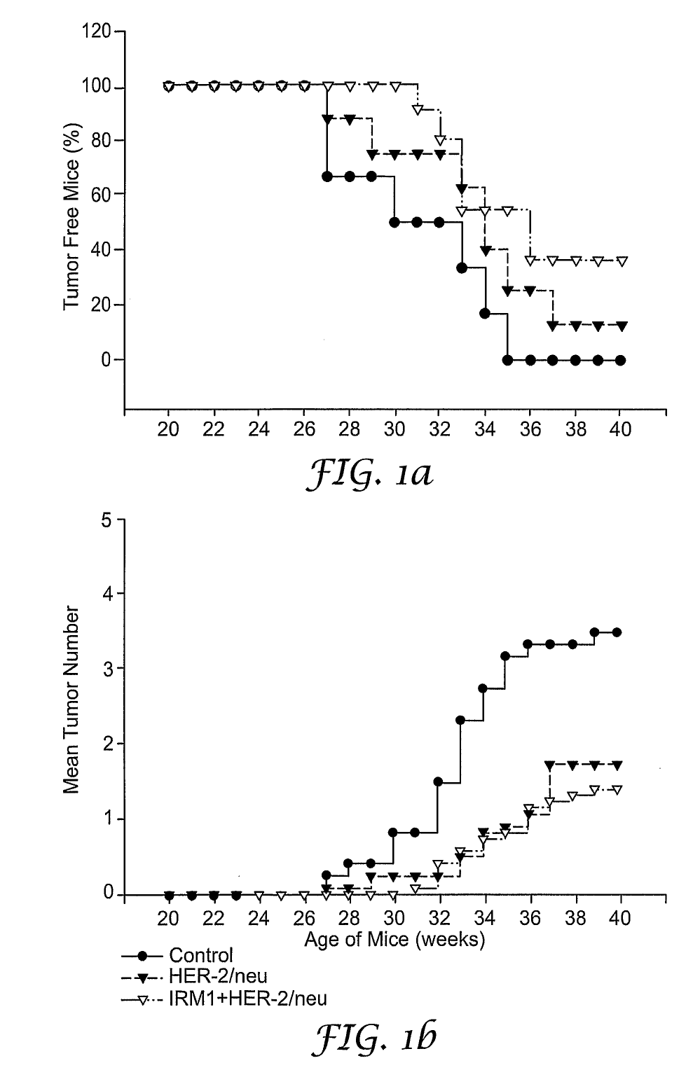

FIG. 1 shows the percentage of tumor-free mice (top) and mean number of palpable mammary carcinomas per mouse (bottom) in mice immunized with vaccine alone or combined with treatment with IRM1.

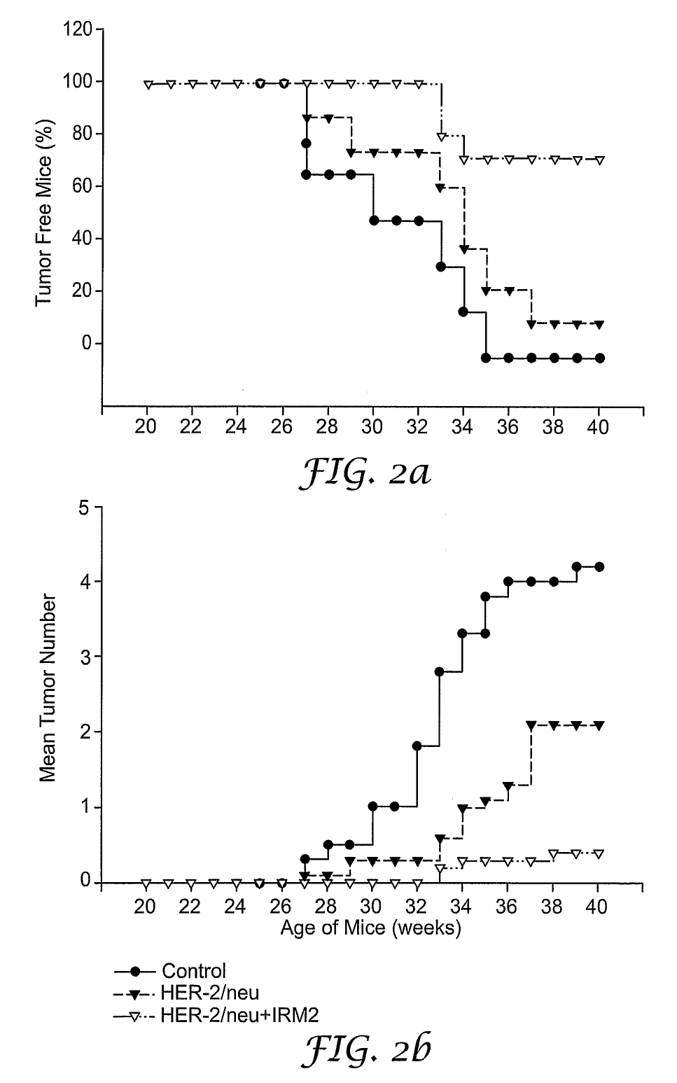

FIG. 2 shows the percentage of tumor-free mice (top) and mean number of palpable mammary carcinomas per mouse (bottom) in mice immunized with vaccine alone or combined with treatment with IRM2.

### Example 2

### Antigen-Specific Cytotoxicity Assay

Animals were grouped and immunized and/or treated as described in Example 1. Spleens were harvested and teased through a 60 micron mesh sieve in Ca²⁺-free and Mg²⁺-free phosphate buffered saline (PBS, GIBCO, Gaithersburg, Md.) solution. Spleen cells were fractionated on lymphocyte M (Cedarlane Laboratories, Ltd., Hornby, Ontario, Canada) and mononuclear cells separated by density gradient centrifugation (500 g, 20 min.). Cells from the interface of the gradients were washed twice with PBS and resuspended in RPMI 1640 (Life Technologies, Inc., Gaithersburg, Md.) containing penicillin (100 U/mL) and streptomycin (100 μg/mL).

Splenocytes were incubated at 37° C. and 5% CO₂ in RPMI medium containing 10% fetal calf serum (FCS, Life Technologies, Inc., Gaithersburg, Md.) in the presence of N202.1A tumor cells (Nanni et al., Int. J. Cancer (2000), vol. 87, pp. 186-194) as stimulators (20:1 ratio stimulators:lymphocytes) for 5 days.

A stock solution of carboxyfluorescein diacetate (c'FDA, Molecular Probes, Inc. Eugene, Oreg.) (20 mg/mL acetone, stored a −20° C.) was diluted in PBS to give a final concentration of 75 μg/mL. N202.1A tumor cells were washed twice with PBS and then labeled with c'FDA by resuspending the cells in 1 mL working solution and incubating at 37° C. in a humidified 5% CO₂ incubator for 30 minutes. Target cells were then washed three times in PBS containing 1% BSA (Sigma Chemical Co., St. Louis, Mo.) suspended in RPMI+10% FCS at a concentration of 1×10⁵ cells/mL.

1×10⁴ c'FDA-labeled tumor target cells were incubated with effector spleen cells in 200 μL total volume in 96-well round microtiter plates (Nunc A/S, Roskilde, Denmark). Effector:target cells ratios ranging from 100:1 to 12.5:1 were tested in triplicate. The plates were kept at 37° C. in a humidified 5% CO₂ incubator for three hours, then centrifuged at 700 g for five minutes. The supernatant was separated from the cellular fraction by rapidly inverting the plates and flicking the supernatant out. 100 μL of 1% Triton X100 in 0.05 M borate buffer, pH 9.0 was added to each well. The plate was kept for 20 hours at 4° C. to allow for solubilization. Plates were read for fluorescence with a 1420 VICTOR² multilabel counter (PerkinElmer Life and Analytical Sciences, Inc., Boston, Mass.). The percentage of specific lysis (i.e., antigen-specific cytotoxicity) was calculated as follows:

$$\% Specific Lysis = \frac{( {F}_{med} - {F}_{exp}}{{F}_{med}} \times 100$$

where,
F represents the fluorescence of the solubilized cells after the supernatant is removed;
F_{med}=F from target incubated in medium alone; and
Fₑₓₚ=F from target incubated with effector cells.

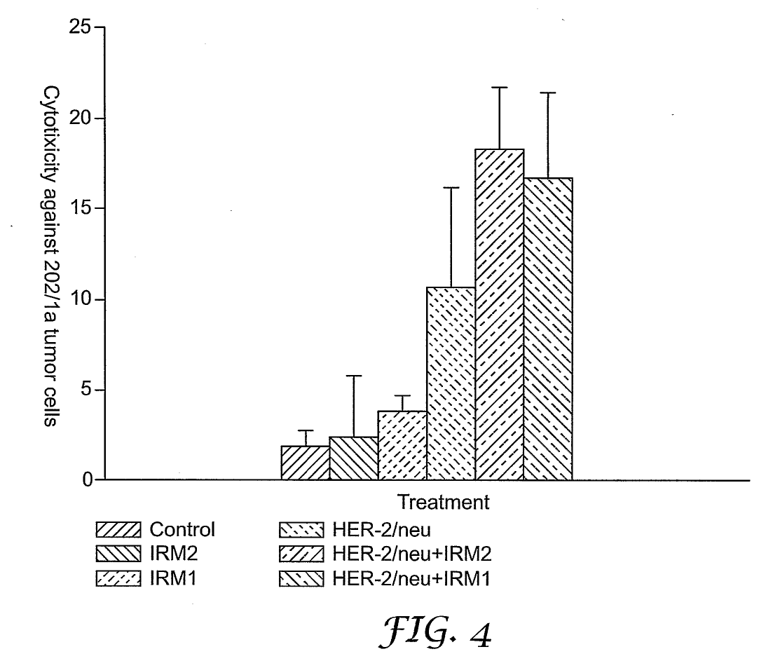

Results are summarized in FIG. 4.

### Example 3

### Antigen-Specific Humoral Immunity

Animals were grouped and immunized and/or treated as described in Example 1. Two weeks after the immunization period was completed, sera were harvested from control and experimental animals. Sera were stored at −80° C. and successively analyzed by flow cytometry. 2×10⁵ N202.1A cells, which express high levels of tumor specific antigen p185^{neu}, were washed twice with cold PBS supplemented with 2% BSA and 0.5% sodium azide (PBS-azide-BSA). Cells were then stained in a standard indirect immunofluorescence procedure using 50 μL of control or immune sera diluted 1:10 in PBS-azide-BSA. A fluorescein-conjugated rabbit anti-mouse Ig (EMD Biosciences, Inc., San Diego, Calif.) was used as the secondary antibody. The cells were resuspended in Isoton II and evaluated through a COULTER EPICS XL (Beckman Coulter, Inc., Fullerton, Calif.) flow cytometer. The N202.1A binding potential (Sbp), a measure of antigen-specific humoral immune response, of the sera were calculated as follows:
Sbp=[(%_{T})(fluorescent mean)]−[(%_{C})(fluorescent mean)]×serum dilution
where,
%_{T} is the percent of positive cells in test serum; and
%_{C} is the percent of positive cells in control serum.

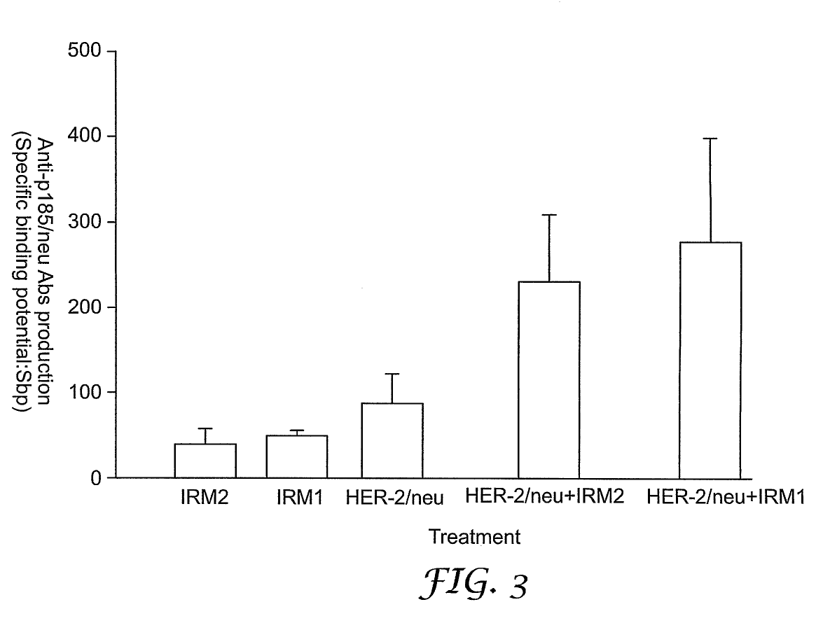

Results are summarized in FIG. 3.

### Example 4

### Intracellular Cytokine Staining

Splenocytes were obtained as described in Example 2 and were incubated overnight at 37° C. and 5% CO₂ in RPMI medium containing 10% FCS in the presence of N202.lA tumor cells as stimulators (20:1 ration stimulators:lymphocytes). Cells were harvested and stained in PBS buffer containing 5% FCS and 0.01% NaN3, with PE-conjugated anti-CD4 or anti-CD8 monoclonal antibodies (BD Biosciences, Becton, Dickinson and Co., San Jose, Calif.). Cells were then fixed in 0.2% formaline, successively stained in a PBS buffer containing 5% FCS and 0.05% formaline with FITC conjugated anti-IL10, anti-IL-12, or anti-IFN-γ (BD Biosciences). Staining was evaluated by a COULTER EPICS XL flow cytometer (Beckman Coulter, Inc., Fullerton, Calif.).

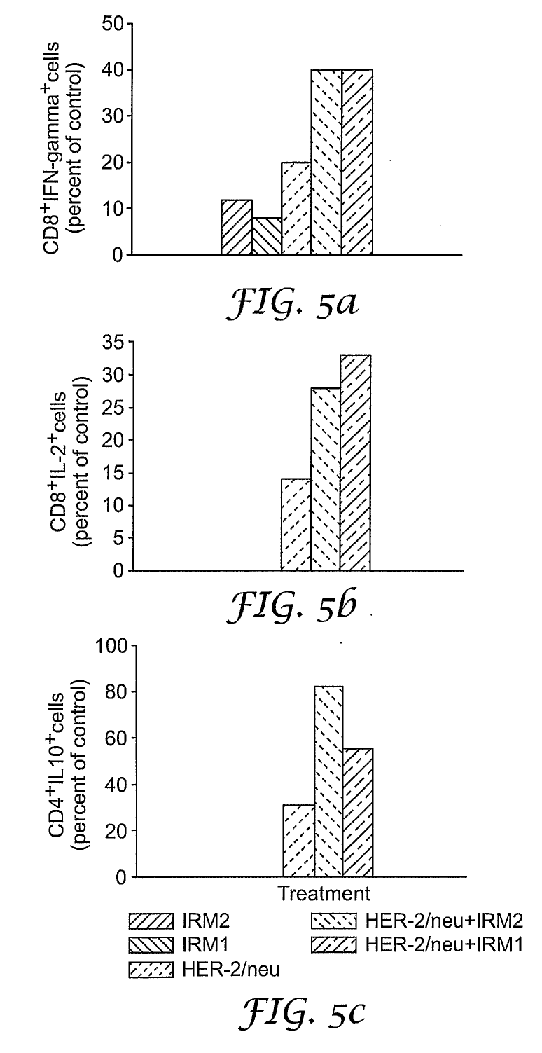

Results are shown in FIG. 5.

### Example 5

Rhesus macaques are immunized in the upper left arm with 50 μg or 100 μg of the pCMV-ECD-TM vaccine, prepared and delivered as described above, on day 2, 30, and 58. At the site of immunization, animals treated with IRM compounds receive intradermal injections containing 0.5 mg/kg of IRM1, or 0.05 mg/kg, 0.5 mg/kg, or 5 mg/kg of IRM3, IRM4, IRM5, or IRM6, or 50 mg/kg of IRM5, dissolved in PBS. Those receiving IRM compound are treated every three days during the period starting on day 0. On days 16, 45, and 72 blood is collected and the number of IFN-γ producing cells is measured by ELISPOT. The number of IFN-γ producing cells will vary in an IRM dose-dependent manner.

### Example 6

Rhesus macaques are grouped and immunized and/or treated as described in Example 5. Two weeks after the immunization period is completed, sera are harvested from control and experimental animals. Sera are stored at −80° C. and successively analyzed by flow cytometry. 2×10⁵ SK-BR-3 cells (ATCC, Mannasas, Va.), which express high levels of tumor specific antigen Her-2, are washed twice with cold PBS supplemented with 2% BSA and 0.5% sodium azide (PBS-azide-BSA). Cells are then stained in a standard indirect immunofluorescence procedure using 50 μL of control or immune sera diluted 1:10 in PBS-azide-BSA. A fluorescein isthothiocyanate-conjugated donkey anti-human IgG (H+L) (Jackson ImmunoResearch Labs, Inc., West Grove, Pa.) is used as the secondary antibody. The cells are resuspended in flow cytometry staining buffer (Biosource International, Carmarillo, Calif.) and evaluated through a FACSCalibur (BD Biosciences, San Jose, Calif.) flow cytometer. The SK-BR-3 binding potential (Sbp), a measure of antigen-specific humoral immune response, of the sera are calculated as follows:
Sbp=[(%_{T})(fluorescent mean)]−[(%_{C})(fluorescent mean)]×serum dilution
where,
%_{T} is the percent of positive cells in test serum; and
%_{C} is the percent of positive cells in control serum.

Spb will vary in an IRM dose-dependent manner.

### Example 7

Animals were treated as in Example 1 for each of the following groups: (1) Immunized with pCMV-ECD-TM, not treated with IRM (HER-2/neu); (2) Immunized with pCMV-ECD-TM, treated with IRM2 (IRM+HER-2/neu); or (3) untreated (Control). Two weeks after the immunization period was completed, sera were harvested from the animals and pooled among animals receiving the same treatment.

150 μL of pooled serum was injected into eight-week old animals (5 animals/treatment serum). Twenty-four hours after administration of the serum, each mouse was challenged with subcutaneously with 10⁵ N202/1A tumor cells and monitored to register the development of tumors.

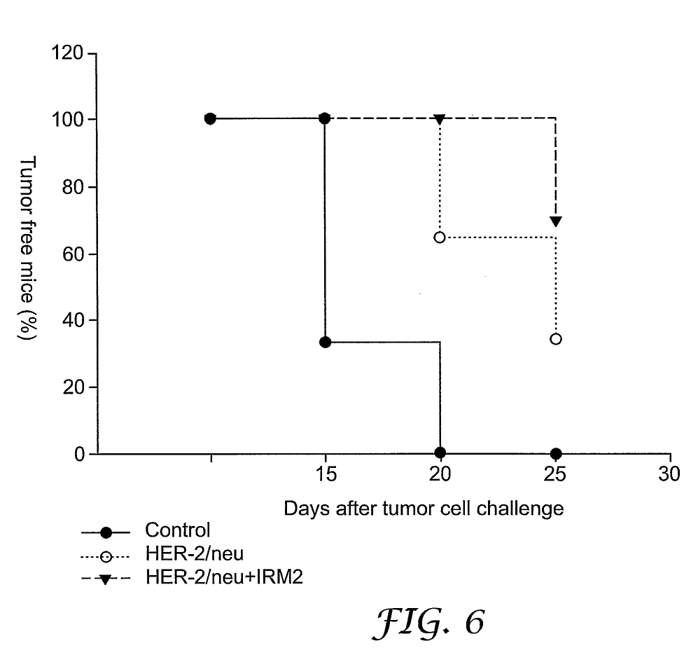

Results are shown in FIG. 6. A greater percentage of animals treated with serum from mice immunized with pCMV-ECD-TM remained tumor free compared with the control mice. An even greater percentage of mice treated with serum from mice immunized with pCMV-ECD-TM and IRM2 remained tumor free throughout the course of monitoring.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

1-42. (canceled)

43. A DNA vaccine composition that comprises:
an expression vector that encodes a clinically relevant breast cancer-associated antigenic peptide; and
an IRM compound.

44. The composition of claim 43 wherein the clinically relevant breast cancer-associated antigenic peptide comprises at least a portion of human HER-2 protein.

45. The composition of claim 43 wherein the clinically relevant breast cancer-associated antigenic peptide comprises at least a portion of rat p185^{neu} protein.

46. The composition of claim 43 wherein the clinically relevant breast cancer-associated antigenic peptide comprises at least a portion of mouse Her-2/neu protein.

47. The composition of claim 43 wherein the clinically relevant breast cancer-associated antigenic peptide comprises at least a portion of mammaglobulin-A.

48. The composition of claim 43 wherein the clinically relevant breast cancer-associated antigenic peptide comprises at least a portion of MUC1.

49. The composition of claim 43 wherein the IRM compound comprises an imidazoquinoline amine.

50. The composition of claim 49 wherein the IRM compound comprises 1-(2-methylpropyl)-1H-imidazo[4,5-c]quinolin-4-amine.

51. The composition of claim 49 wherein the IRM compound comprises 4-amino-α,α,2-trimethyl-1H-imidazo[4,5-c]quinoline-1-ethanol.

52. The composition of claim 43 wherein the IRM compound comprises a TLR8-selective agonist.

53. The composition of claim 43 wherein the expression vector and the IRM compound are provided in separate formulations.

54. A DNA vaccine adjuvant composition comprising:
a TLR8-selective agonist in an amount effective to increase the efficacy of a DNA composition.

55. The composition of claim 54 wherein the adjuvant comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydro imidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

56. The composition of claim 55 wherein the IRM compound comprises a thiazoloquinoline amine.

57. The composition of claim 56 wherein the IRM compound comprises 2-propylthiazolo[4,5-c]quinolin-4-amine.

58. The composition of claim 56 wherein the IRM compound comprises 2-propyl-7-(pyridin-3-yl)-thiazolo[4,5-c]quinolin-4-amine.

59. The composition of claim 56 wherein the IRM compound comprises N-[3-(4-amino-2-propylthiazolo[4,5-c]quinolin-7-yl)phenyl]methanesulfonamide.

60. The composition of claim 56 wherein the IRM compound comprises [3-(4-amino-2-propylthiazolo[4,5-c]quinolin-7-yl)phenyl]methanol.

61. A DNA vaccine comprising the composition of claim 54.

62. A method of generating an immune response against a clinically relevant breast cancer associated antigenic peptide in a subject, the method comprising:
immunizing the subject with a vaccine that comprises:
an expression vector that encodes a clinically relevant breast-associated antigenic peptide, and
an IRM compound.

63. The method of claim 62 wherein the antigenic peptide is a hepatocellular cancer-associated peptide, a cervical cancer-associated peptide, a melanoma-associated peptide, a lung cancer-associated peptide, a colon cancer-associated peptide, a breast cancer-associated peptide, a pancreatic cancer-associated peptide, or an ovarian cancer-associated peptide.

64. The method of claim 62 wherein the cancer-associated antigenic peptide comprises Her-2/neu, mammaglobulin-A, MUC1, alphafetoprotein, HPV E6, HPV E7, TRP-1, or VEGF2.

65. The method of claim 62 wherein the IRM compound comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

66. The method of claim 65 wherein the compound comprises a thiazoloquinoline amine.

67. A method of generating an immune response against a clinically relevant cancer-associated antigenic peptide in a subject, the method comprising:
immunizing, a subject with a vaccine that comprises
an expression vector that encodes a clinically relevant cancer-associated antigenic peptide, and
a compound that is a TLR8-selective agonist.

68. The method of claim 67 wherein the antigenic peptide is a hepatocellular cancer-associated peptide, a cervical cancer-associated peptide, a melanoma-associated peptide, a lung cancer-associated peptide, a colon cancer-associated peptide, a breast cancer-associated peptide, a pancreatic cancer-associated peptide, or an ovarian cancer-associated peptide.

69. The method of claim 67 wherein the cancer-associated antigenic peptide comprises Her-2/neu, mammaglobulin-A, MUC1, alphafetoprotein, HPV E6, HPV E7, TRP-1, or VEGF2.

70. The method of claim 67 wherein the IRM compound comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine a tetrahydroimidazonaphthyridine amine, an oxazoloquinoline amine a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

71. The method of claim 70 wherein the IRM compound comprises an imidazoquinoline amine, a tetrahydroimidazoquinoline amine, an imidazopyridine amine, a 1,2-bridged imidazoquinoline amine, a 6,7-fused cycloalkylimidazopyridine amine, an imidazonaphthyridine amine, a tetrahydroimidazonaphthyridine amine, an oxazoloquinoline amine, a thiazoloquinoline amine, an oxazolopyridine amine, a thiazolopyridine amine, an oxazolonaphthyridine amine, a thiazolonaphthyridine amine, a pyrazolopyridine amine, a pyrazoloquinoline amine, a tetrahydropyrazoloquinoline amine, a pyrazolonaphthyridine amine, or a tetrahydropyrazolonaphthyridine amine.

72. A method of treating breast cancer in a subject, the method comprising:
administering to the subject an expression vector that encodes a clinically relevant breast cancer-associated antigenic peptide in an amount effective to generate an immune response against the clinically relevant breast cancer-associated antigenic peptide; and
administering to the subject an IRM compound in an amount effective to potentiate the immune response to the clinically relevant breast cancer-associated antigenic peptide.

73. The method of claim 72 wherein the breast cancer comprises invasive breast cancer or ductal carcinoma in situ.

74. The method of claim 72 wherein treating breast cancer comprises generating humoral antibodies against the clinically relevant breast cancer-associated antigenic peptide, reducing the size of a breast cancer tumor reducing the number of breast cancer tumors, or decreasing the likelihood that ductal carcinoma in situ progresses to invasive breast cancer.

75. The method of claim 72 wherein treating the cancer comprises generating humoral antibodies against the clinically relevant cancer-associated antigenic peptide reducing the size of a tumor, reducing the number of tumors, delaying the incidence of tumors, prolonging the expected lifespan of the subject, or generating antigen-specific cytotoxic T lymphocytes.

76. A method of treating cancer in a subject, the method comprising:
administering to the subject an expression vector that encodes a clinically relevant cancer-associated antigenic peptide in an amount effective to generate an immune response against the clinically relevant cancer-associated antigenic peptide; and
administering to the subject a TLR8-selective agonist in an amount effective to potentiate the immune response to the clinically relevant cancer-associated antigenic peptide.

77. The method of claim 76 wherein the cancer comprises hepatocellular cancer, cervical cancer, melanoma, lung cancer, colon cancer, breast cancer, pancreatic cancer, or ovarian cancer.

78. The method of claim 76 wherein the cancer-associated antigenic peptide comprises Her-2/neu, mammaglobulin-A, MUC1, alphafetoprotein, HPV E6, HPV E7, TRP-1, or VEGF2.

79. The method of claim 76 wherein treating the cancer comprises generating humoral antibodies against the clinically relevant cancer-associated antigenic peptide, reducing the size of a tumor, reducing the number of tumors, delaying the incidence of tumors, prolonging the expected lifespan of the subject or generating antigen-specific cytotoxic T lymphocytes.

80. A method of preparing a cancer treatment composition, the method comprising:
administering to the subject an expression vector that encodes a clinically relevant cancer-associated antigenic peptide in an amount effective to generate an immune response against the clinically relevant cancer-associated antigenic peptide;
administering to the subject an IRM compound in an amount effective to potentiate the immune response to the clinically relevant cancer-associated antigenic peptide;
permitting the subject to generate a serum immune response to the clinically relevant cancer-associated antigenic peptide; and
collecting at least a portion of the subject's serum.

81. The method of claim 80 wherein the cancer comprises hepatocellular cancer, cervical cancer, melanoma, lung cancer, colon cancer, breast cancer, pancreatic cancer, or ovarian cancer.

82. The method of claim 80 wherein the cancer-associated antigenic peptide comprises Her-2/neu, mammaglobulin-A, MUC1, alphafetoprotein, HPV E6, HPV E7, TRP-1, or VEGF2.

83. A method of treating cancer in a subject, the method comprising:
administering to a mammal an expression vector that encodes a clinically relevant cancer-associated antigenic peptide in an amount effective to generate an immune response against the clinically relevant cancer-associated antigenic peptide;
administering to the mammal an IRM compound in an amount effective to potentiate the immune response to the clinically relevant cancer-associated antigenic peptide;
permitting the mammal to generate a serum immune response to the clinically relevant cancer-associated antigenic peptide;
collecting at least a portion of the mammal's serum; and
administering at least a portion of the mammal's serum to the subject in an amount effective to treat the cancer.

84. The method of claim 83 wherein the potion of the mammal's serum comprises an antibody against the clinically relevant cancer-associated antigen.
